(19) United States
(12) Patent Application Publication
(10) Pub. No.: US 2008/0281410 A1
(43) Pub. Date: Nov. 13, 2008

(54) Method for Production of a Coated Endovascular Device

(75) Inventors: Aleardo Maresta, Ravenna (IT); Antonio Ravaglioli, Faenza (IT)

Correspondence Address: GIFFORD, KRASS, SPRINKLE,ANDERSON & CITKOWSKI, P.C, PO BOX 7021, TROY, MI, 48007-7021, US

(73) Assignee: I.B.S. International Biomedical Systems S.P.A., Triest (IT)

(21) Appl. No.: 12/091,603

(22) PCT Filed: Oct. 26, 2006
(86) PCT No.: PCT/EP2006/067825

§ 371 Date: Apr. 25, 2008

(30) Foreign Application Priority Data
Oct. 28, 2005 (IT) MO2005A000283

(51) Int. Cl. A61F 2/82; A61L 27/30

(52) U.S. Cl. 623/1.46; 427/2.25

(57) ABSTRACT

A method of coating a endovascular device that includes coating of a tubular body's surface by at least a thin layer (s) of a inert and biocompatible titanium based material. This method is performed by the following steps in succession: Deposition of a first Titanium layer (21). First nitrogen treatment of said first titanium layer (21) by transmission of high ionic currents on the substrate (Closed Field UnBalanced Magnetron Sputter Ion Plating) to obtain the transformation of at least a part of said first titanium layer (21) in a first layer of titanium nitride ceramic coating (210). Deposition on this said first layer of titanium nitride ceramic coating (210) of a second titanium layer (22). Second nitrogen treatment of said second titanium layer (22) by transmission of high ionic currents on the substrate (Closed Field UnBalanced Magnetron Sputter Ion Plating) to obtain the transformation of at least a part of said second titanium layer (22) in a second layer of titanium nitride ceramic coating (220).

### FIELD OF INVENTION

The present invention relates to a method for production of a coated endovascular device with the characteristics in claim 1. It's also object of this invention a coated stent with the characteristics in claim 13.

The present invention relates to the cardiologic medical field and more specifically it relates to the realisation of a medical-surgical device for treatment and prevention of ischemic heart condition.

### BACKGROUND OF THE INVENTION

The ischemic heart condition is the most common heart disease in the west countries and it's the main death cause. In the last decades several devices have been studied to try to fight these diseases and achieved results show that stenting procedure is one of the most efficacious solution.

It's a simple technique that avoids the need to make a more difficult surgery—as surgical revascularization.

As known, stent is a substantially cylindrical prosthetic device with an expandable open structure, generally of steel suitable for medical use, that is implanted in the arterial lesion site (stenosis or occlusion).

Said open structure is expanded until its desired dimension, according to arterial diameter, by the well-known balloon-expansion technique that requires the introduction of ballon, on which the stent is crimped, into the vessel and its subsequent inflation. The balloon, during its expansion, increases the stent diameter until the desired dimension, then it is deflated and withdrawn. The stent remains in the position where it's introduced because of the recoil of blood vessel tissues.

Applicants have noticed that well-known technique stents have several problems and that it is possible to improve them regarding several aspects.

The most important problem of coronary angioplasty is in-stent restenosis. It depends on several factors; the most important of them is intimal hyperplasia, that manifests itself by activation of tunica media vasorum smooth muscle cells because of the damage provoked during the stent application. To avoid this problem, generally, cell and tissue growth inhibiting drugs are used and these are attached to the stent surface. The most used technique to do it is coating the stent surface with a polymer whose role is to retain the drug and to release it slowly in time after the stent implantation. The drug can be distributed over the polymer or it can be introduced between two polymeric layers, or it can be incorporated into the polymeric layer. However, in these cases, the drug is not released gradually and constantly from the stent surface, and this can decrease its effect.

In particular, in the case of metallic stent without polymeric coating it's noticed a secondary cause of cellular proliferation caused by chemical-physical interaction between wall vessel and stent material (that includes nickel among its alloy components).

In fact, it is demonstrated that well-known technique stainless steel stents in contact with organic liquids are subjected to corrosive phenomenon that produce release of nickel, chromium and other substances that inside the body could provoke an allergic reaction.

Moreover, hematic biocompatibility problems increase thrombosis risk during the first days after the implantation. For this reason variations of well-known technique stents have been developed, having a coating on their surface that will be in contact with blood and that is realized with anallergic-materials as depleted uranium, silicon carbide, carbon and polymers.

Metallic stents with anallergic coating, however, have other problems. In fact, the use of coating with ionizing radiation emitting materials, as depleted uranium, could produce an important incidence of tardive thrombosis. The use of carbon as coating material is not appropriate because of its cleavage that occurs when the material is subjected to high mechanical stress due to its expansion during stent implant. The recurrent use of silicon carbide, then, proved not to be the most indicated because of its cytotoxycity at high concentrations. At last polymeric coatings do not currently allow to obtain films of thickness lower than 5 μm.

Another problem of the well-known technique is that methods currently used to produce stents don't permit to obtain a perfectly smooth stent surface, necessary to avoid blood flow turbulences that can worsen damage to wall vessel and incidence of restenosis.

In the name of the same applicant a patent application was filed with number M02003A000238

to give a first solution to the previous problems, and relates to a stent with a titanium nitride coating, able not to release allergic substances and not to interact negatively with the body, thus guaranteeing corrosive resistance, chemical stability and high biocompatibility.

### SUMMARY OF THE INVENTION

Aim of the present invention is to improve the results of the previous invention, object of patent application for industrial invention M02003A000238, with the purpose of producing a coated endovascular device with thinner coating layer, that doesn't modify mechanical characteristics and functionality of the same stent.

Another purpose of this invention is the realization of a endovascular device with a surface so smooth to avoid blood flow turbulences and to reduce platelet activation, thus avoiding or reducing considerably the risk of thrombosis.

The endovascular device object of this invention, moreover, is able to be loaded by a drug and to release it in the planned times.

These purposes and others, that will become clear from the following description, are achieved by a endovascular device with the characteristics reported in claim 1. By the term endovascular device in the present invention it is preferably intended, but not limited to, one of the following types of devices:
a graft for abdominal and thoracic aorta and/or iliac arteries.
a coronary stent.
a peripheral stent.
a biliary stent
a renal stent.
a carotid and cerebral stent.

Other characteristics and advantages of the present invention will be described in the following detailed description of a preferred, but not exclusive, endovascular device and of a method to produce it, according to the present invention.

### BRIEF DESCRIPTION OF THE DRAWINGS

This description is given with reference to the enclosed drawings, which are provided purely for indicative purpose and are then non-limiting.

FIG. 1 shows a stent according to the present invention

FIG. 2 shows, by an enlarged scale, part of a section of the stent of FIG. 1, with highlighted coating layers

FIGS. 3, 4, 5, 6, show, in a schematic way, the same part of a transversal section of the stent wall during several operative phases of the coating production.

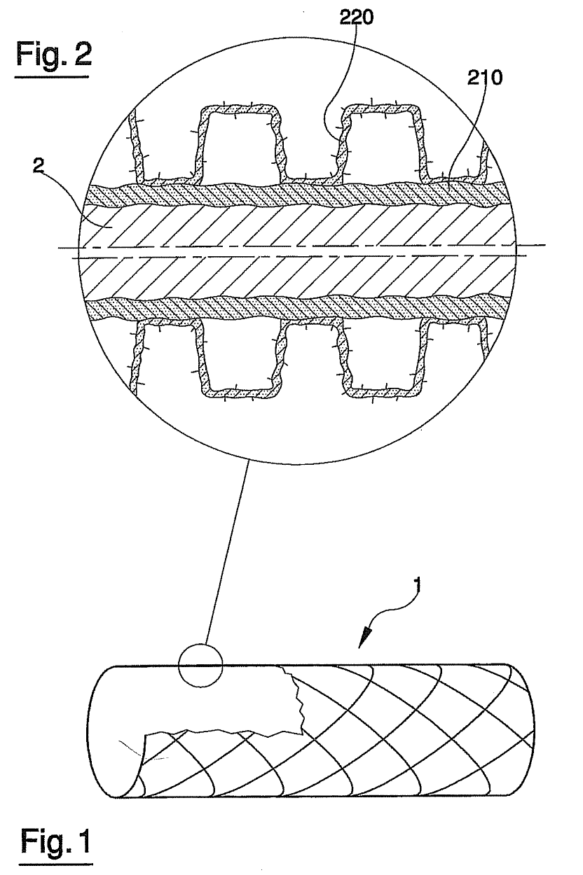

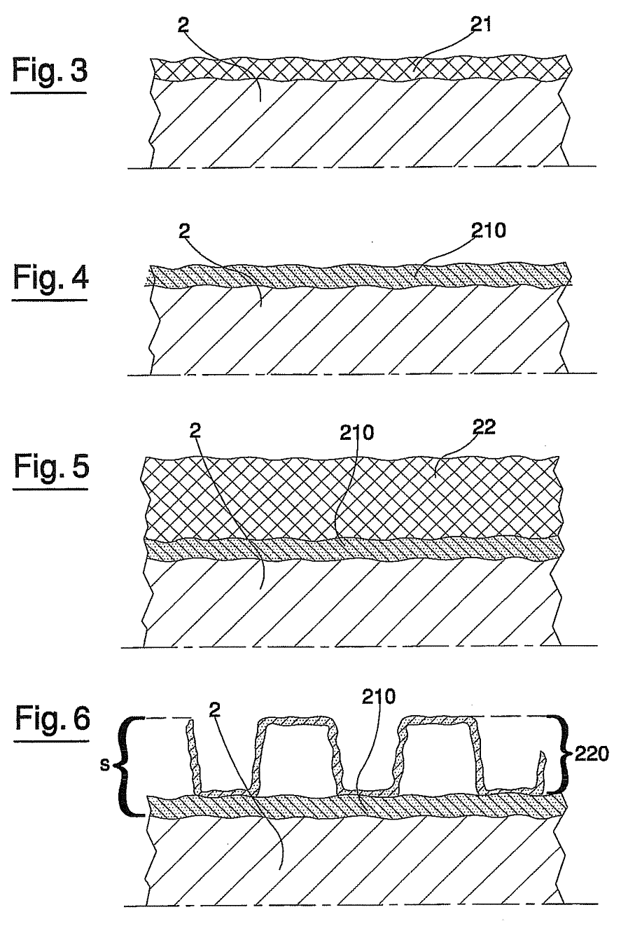

### DETAILED DESCRIPTION

In the following the word stent will be used with the above defined extended meaning.

Referring to the enclosed drawings, it is indicated as 1 a stent according to the present invention.

The stent 1 has a tubular, metallic, flexible and substantially cylindrical body 2 that is made of, for example, a metallic closed net. As an indication, the metallic net can be produced from a stainless steal tube with a circular section by laser cutting. The tubular body 2, generally, is made of a processable material with a high fatigue resistance, as stainless steel 316L. Other kinds of materials are also possible to be used, like the following:
different inert and biocompatible metallic alloy, and in particular of CoCr alloy, as L605 (Co-20Cr-15W-10Ni), Co-28Cr-6Mo, Co-35Ni-20Cr-10Mo, Co-20Cr-16Fe-15Ni-7Mo, because of its major elasticity, that reduces the risk and the entity of micro-fracture during crimping and expansion phases, and the possibility to maintain the same characteristics with a minor thickness.
different inert and biocompatible metallic alloy, and in particular pure Ti or its alloy, as Ti-12Mo-6Zr-2Fe, Ti-15Mo, Ti-3Al-2,5V, Ti-35Nb-7Zr-5Ta, Ti-6AI-4Va, Ti-6AI-7Nb, Ti-13Nb-13Zr.
Nickel-Titanium shape memory alloy(Nitinol).
different inert and biocompatible metallic alloy, and in particular Cr alloy, as Cr-14Ni-2,5Mo, Cr-13Ni-5Mn-2,5Mo, Cr-10Ni-3Mn-2,5Mo.

The tubular body 2 is totally covered by at least an inert and biocompatible coating layer ‘s’, where by the term biocompatible it's indicated a material that is able to interact with wall vessel tissues and hematic blood flow as less as possible, and to not interact negatively with the human body. The thin biocompatible and inert titanium nitride based layer, that covers the whole stent, is obtained after preparation of the tubular substantially cylindrical body 2 made of an expandable metallic net, generally medical stainless steel, by a method that comprises the following operations in succession:
Deposition of a first Titanium layer (21)
First nitrogen (N) treatment of said first titanium (Ti) layer (21) by transmission of high ionic currents on the substrate (Closed Field UnBalanced Magnetron Sputter Ion Plating) aimed to obtain the transformation of at least a part of said first titanium layer (21) in a first layer of titanium nitride (TiN) ceramic coating (210)
Deposition on this said first layer of titanium nitride (TiN) ceramic coating (210) of a second titanium (Ti) layer (22)
A second nitrogen (N) treatment of said second titanium (Ti) layer (22) by
transmission of high ionic currents on the substrate (Closed Field UnBalanced Magnetron Sputter Ion Plating) aimed to obtain the transformation of at least a part of said second titanium (Ti) layer (22) in a second layer of titanium nitride (TiN) ceramic coating (220).

The first titanium layer 21 has preferably a thickness of about 100 nm.

The first nitrogen treatment of the first titanium layer 21 is aimed to transform at least a part of the said first titanium layer 21 into a compact ceramic coating made of titanium nitride 210.

The second nitrogen treatment of said second titanium layer (22) by transmission of high ionic currents on the substrate (Closed Field UnBalanced Magnetron Sputter Ion Plating) is aimed to obtain the transformation of the whole said second titanium layer 22 into a second ceramic coating layer fully made of titanium nitride 220.

The first layer, formed at least in part by titanium nitride, makes the second treatment safe, avoiding it to get into direct contact with the external surface of the tubular cylindrical body 2.

The second treatment is made so that at least the external part of the whole ceramic coating made of titanium nitride (TiN) has a morphology that is of the same kind of that represented in FIG. 2. In particular this morphology is characteristic of the whole ceramic coating made of porous titanium nitride 220.

The thin inert and biocompatible titanium layer ‘s’ (that is made of titanium nitride wholly or almost wholly) that covers the stent has a thickness of about 1-2 μm, and preferably of about 1.5 μm.

The external surface of the ceramic coating made of titanium nitride (TiN) is characterised by a pre-established porosity aimed to increase the retention of a layer, even if a monomolecular layer, of drug.

More specifically, the mentioned nitrogen treatments are made using an ionic deposition system made by at least one magnetron.

The successive step of this coating method is characterised by a deposition of an anti restenosis drug over the external surface of the said biocompatible material that covered the tubular body 2.

Before this step implementation, a preliminary phase aimed to remove any contaminations from the tubular body 2 to be coated is necessary.

In particular, treatment operations for titanium deposition are made by at least one magnetron and comprises the following steps:
The insertion of the tubular body 2 into a vacuum chamber
The insertion of at least a titanium element into said vacuum chamber
The insertion of a noble gas into said vacuum chamber
The bombardment by electrons generated by at least one magnetron of noble gas atoms to obtain noble gas ions
The bombardment by said noble gas ions of said titanium element to obtain titanium ions
The induction of a potential difference between tubular body 2 and said vacuum chamber to obtain the deposition of said titanium ions over the tubular body.

Then, the titanium nitride deposition is produced by a successive phase during which nitrogen gas is introduced into said vacuum chamber to obtain titanium nitride.

It's important to notice that titanium nitride coating of the stent has a lower wettability for proteins than stainless steel stent surface of the well-known technique.

This coating ensures that there is no release of toxic ions from the same coating and from the underlying steel.

With the above described method it is possible to obtain coatings made of titanium compounds with a medium low thickness (about 1.5 μm) and with a very thin and smooth structure that ensures a high resistance to the mechanical stress generated during stent implantation, without modifying the stent elastic deformability.

At the end of the coating treatment the stent is coated by a thin biocompatible inert titanium nitride based layer that includes:
A first coating ceramic layer made of titanium nitride (210) that is into contact and bounded with the external-stent surface
A second titanium based layer bounded directly with said first ceramic coating layer made of titanium nitride (210) and said second layer is made of, at least in part, a second ceramic titanium nitride coating layer.

The first ceramic titanium nitride coating layer (210) is compact, differently from the second layer that is directly bounded to it, which is wholly composed by titanium nitride and has a pre-established porosity and a columnar morphology. The thin inert biocompatible titanium nitride based layer that covers the whole stent has a thickness of about 1-2 μm.

Finally, the particular kind of the deposited titanium nitride crystal structure allows the application of drugs over the same coating, their release in the body according to fixed time and the possibility to use a monomolecular polymeric activating thin layer (for example polymeric micelles as lyposomes).

Another possibility is to put over the stent an endothelial cell layer to facilitate a faster blood vessel endothelialisation and to reduce the incidence of acute and sub-acute thrombosis after implantation, thus reducing restenosis entity.

Optionally the procedure subject of the invention comprises a preliminary polishing step aimed to eliminate any kind of surface contamination and/or defects due to laser cutting, like lateral re-fused material successive to thermal explosion, from the tubular body to be coated.

In addition, said preliminary polishing step can be operated by alumina powder (Al 203) and if this is not sufficient, it is possible to operate using a chemical attack with 3D photolithography methods and structures.

Furthermore, this said preliminary polishing step can be also chemical, sand, electrolytic and/or electrochemical polishing.

1. A method for the realization of a coated endovascular device comprising at least the steps of:
preparation of a substantially cylindrical tubular body (2) made of an inert and biocompatible metal or metallic alloy selected from the group consisting of stainless steel, CoCr alloy, Ti or its alloy, Cr alloy;
coating of said tubular body surface with at least one thin inert biocompatible titanium based layer (s), said coating being produced according to the following successive steps:
I. deposition of a first Titanium (Ti) layer (21);
II. first nitrogen (N) treatment of said first titanium (Ti) layer (21) by transmission of high ionic currents on the substrate (Closed Field UnBalanced Magnetron Sputter Ion Plating) aimed to obtain the transformation of at least a part of said first titanium layer (21) in a first layer of titanium nitride (TiN) ceramic coating (210);
III. deposition on this said first layer of titanium nitride (TiN) ceramic coating (210) of a second titanium (Ti) layer (22);
IV. a second nitrogen (N) treatment of said second titanium (Ti) layer (22) by transmission of high ionic currents on the substrate (Closed Field UnBalanced Magnetron Sputter Ion Plating) aimed to obtain the transformation of at least a part of said second titanium (Ti) layer (22) in a second layer of titanium nitride (TiN) ceramic coating (220).

2. The method of claim 1, wherein the said first nitrogen (N) treatment of the said first titanium (Ti) layer (21) is aimed to transform at least a part of the said first titanium layer (21) into a compact ceramic titanium nitride coating (210).

3. The method of claim 1, wherein the second nitrogen treatment of the said second titanium layer (22), made by transmission of high ionic currents on the substrate (Closed Field UnBalanced Magnetron Sputter Ion Plating) is aimed to transform the whole said second titanium layer (22) into a second ceramic porous titanium nitride layer (220).

4. The method of claim 1, wherein the said first titanium (Ti) layer thickness is about 100 nm.

5. The method of claim 1, wherein the said thin inert biocompatible titanium nitride based layer (s) that coated wholly the endovascular device has a thickness of about 1-2 μm.

6. The method of claim 1, wherein at least the external part of the said ceramic titanium nitride (TiN) coating has a columnar morphology.

7. The method of claim 1, wherein at least the external surface of the said ceramic titanium nitride (TiN) coating is characterized by a predetermined porosity.

8. The method of claim 1, wherein said nitrogen treatments are produced by the use of a ionic deposition system made by at least a magnetron.

9. The method of claim 1, characterized by the fact that further comprises a subsequent step of anti restenosis drug deposition over the external porous surface of the said biocompatible layer (s) that covered the tubular body.

10. The method of claim 1, wherein the said endovascular device is a graft for abdominal and thoracic aorta and/or iliac arteries.

11. The method of claim 1, wherein the said endovascular device is a coronary stent.

12. The method of claim 1, wherein the said endovascular device is a peripheral stent.

13. The method of claim 1, wherein the said endovascular device is a biliary stent.

14. The method of claim 1, wherein the said endovascular device is a renal stent.

15. The method of claim 1, wherein the said endovascular device is a carotid and cerebral stent.

16. The method of claim 1 wherein the in said endovascular device the substantially cylindrical tubular body (2) is made of an inert and biocompatible 316L steel.

17. The method of claim 1, wherein the in said endovascular device the substantially cylindrical tubular body (2) is made of an inert and biocompatible CoCr alloy selected from the group consisting of L605 (Co-20Cr-15W-10Ni), Co-28Cr-6Mo, Co-35Ni-20Cr-10Mo, Co-20Cr-16Fe-15Ni-7Mo.

18. The method of claim 1, wherein the in said endovascular device the substantially cylindrical tubular body (2) is made of an inert and biocompatible Ti or its alloy selected from the group consisting of Ti-12Mo-6Zr-2Fe, Ti-15Mo, Ti-3Al-2,5V, Ti-35Nb-7Zr-5Ta, Ti-6Al-4Va, Ti-6Al-7Nb, Ti-13Nb-13Zr.

19. The method of claim 1, wherein the in said endovascular device the substantially cylindrical tubular body (2) is made of Nickel-Titanium shape memory alloy (Nitinol).

20. The method of claim 1, wherein the in said endovascular device the substantially cylindrical tubular body (2) is made of an inert and biocompatible Cr alloy selected from the group consisting of Cr-14Ni-2,5Mo, Cr-13Ni-5Mn-2,5Mo, Cr-10Ni-3Mn-2,5Mo.

21. The method of claim 1, wherein it further comprises a preliminary polishing step aimed to eliminate any kind of surface contamination and defects due to laser cutting, like lateral re-fused material successive to thermal explosion, from the tubular body to be coated.

22. The method of claim 21, wherein the said preliminary polishing step is operated by alumina powder (Al 203) and if this is not sufficient, it is possible to operate using a chemical attack with 3D photolithography methods and structures.

23. The method of claim 21, wherein the said preliminary polishing step can be also chemical, sand, electrolytic and/or electrochemical polishing.

24. The method of claim 1, wherein said treatment operations are made by the use of at least a magnetron and that comprises the following steps:
the insertion of the tubular body (2) into a vacuum chamber;
the insertion of at least a titanium element into said vacuum chamber;
the insertion of a noble gas into said vacuum chamber;
the bombardment by electrons generated by at least a magnetron of noble gas atoms to obtain noble gas ions;
the bombardment by said noble gas ions of said titanium element to obtain titanium ions;
the induction of a potential difference between tubular body (2) and said vacuum chamber to obtain the deposition of said titanium ions over tubular body.

25. The method of claim 24, wherein the procedure further comprises a phase of the nitrogen gas introduction into said vacuum chamber aimed to obtain titanium nitride.

26. A coated endovascular device comprising
a tubular substantially cylindrical body (2) made of an inert and biocompatible metal or metallic alloy selected from the group consisting of stainless steel, CoCr alloy, Ti or its alloy, Cr alloy,
having said tubular substantially cylindrical body bounded on its external surface at least one thin biocompatible inert titanium based layer (s) that comprises:
a first coating ceramic layer made of at least in part, titanium nitride (210) that is into contact and bounded with the external device surface;
a second titanium based layer bounded directly with said first ceramic coating layer made of titanium nitride (210) and said second layer is made of, at least in part, a second ceramic titanium nitride coating layer (220).

27. The coated endovascular device of claim 26, wherein that said first ceramic titanium nitride coating is compact.

28. The coated endovascular device of claim 26, wherein that said second titanium based layer bounded directly on said first ceramic titanium nitride (210) is wholly formed by titanium nitride.

29. The coated endovascular device of claim 26, wherein said first titanium (Ti) layer (21) thickness is about 100 nm.

30. The coated endovascular device of claim 26, wherein said second titanium based layer has a columnar morphology and a pre-established porosity.

31. The coated endovascular device of claim 26, wherein said thin inert biocompatible titanium nitride based coating layer (s) has a thickness of about 1-2 μm.

32. The coated endovascular device of claim 26, wherein the substantially cylindrical tubular body (2) is made of an inert and biocompatible 316L steel.

33. The coated endovascular device of claim 26, wherein the substantially cylindrical tubular body (2) is made of an inert and biocompatible CoCr alloy selected from the group consisting of L605 (Co-20Cr-15W-10Ni), Co-28Cr-6Mo, Co-35Ni-20Cr-10Mo, Co-20Cr-16Fe-15Ni-7Mo.

34. The coated endovascular device of claim 26, wherein the substantially cylindrical tubular body (2) is made of an inert and biocompatible Ti or its alloy selected from the group consisting of Ti-12Mo-6Zr-2Fe, Ti-15Mo, Ti-3Al-2,5V, Ti-35Nb-7Zr-5Ta, Ti-6Al-4Va, Ti-6Al-7Nb, Ti-13Nb-13Zr.

35. The coated endovascular device of claim 26, wherein the substantially cylindrical tubular body (2) is made of an inert and biocompatible Nickel-Titanium shape memory alloy (Nitinol).

36. The coated endovascular device of claim 26, wherein the substantially cylindrical tubular body (2) is made of an inert and biocompatible Cr alloy selected from the group consisting of Cr-14Ni-2,5Mo, Cr-13Ni-5Mn-2,5Mo, Cr-10Ni-3Mn-2,5Mo.

37. The coated endovascular device of claim 26, further comprising an anti restenosis drug deposited over the external porous surface of the said biocompatible layer(s) that covered the tubular body.

38. The coated endovascular device of claim 26, wherein said endovascular device is a graft for abdominal and thoracic aorta and/or iliac arteries.

39. The coated endovascular device of claim 26, wherein said endovascular device is a coronary stent.

40. The coated endovascular device of claim 26, wherein said endovascular device is a peripheral stent.

41. The coated endovascular device of claim 26, wherein said endovascular device is a biliary stent.

42. The coated endovascular device of claim 26, wherein said endovascular device is a renal stent.

43. The coated endovascular device of claim 26, wherein said endovascular device is a carotid and cerebral stent.
